(19) United States
(12) Patent Application Publication
(10) Pub. No.: US 2026/0008993 A1
(43) Pub. Date: Jan. 8, 2026

(54) L-HOMOSERINE HIGH-YIELD STRAIN, CONSTRUCTION METHOD THEREFOR, AND USE THEREOF

(71) Applicants: NANJING ASCEND BIOTECHNOLOGY INSTITUTE CO., LTD, Nanjing (CN); NANJING SHENGDE BIOTECH CO., LTD, Nanjing (CN)

(72) Inventors: Shasha ZHANG, Nanjing (CN); Luqiu SHI, Nanjing (CN); Guizhen SU, Nanjing (CN)

(73) Assignees: NANJING ASCEND BIOTECHNOLOGY INSTITUTE CO., LTD, Nanjing, JS (CN); NANJING SHENGDE BIOTECH CO., LTD, Nanjing, JS (CN)

(21) Appl. No.: 18/993,591

(22) PCT Filed: Jul. 28, 2022
(86) PCT No.: PCT/CN2022/108501

§ 371 Date: Jan. 13, 2025

(30) Foreign Application Priority Data
Jul. 13, 2022 (CN) CN202210853986.1

(51) Int. Cl. C12N 1/20; C12N 9/04; C12N 9/12; C12N 15/70; C12R 1/19

(52) U.S. Cl. CPC C12N 1/20; C12N 9/0006; C12N 9/1217; C12N 15/70; C12N 2800/101; C12R 2001/19; C12Y 101/01003; C12Y 207/02004

(57) ABSTRACT

The present disclosure provides a recombinant Escherichia coli strain modified by metabolic engineering means and a method for producing L-homoserine by using the same. The strain, designated as Escherichia coli having a strain number of 13-XA, is deposited in China General Microbiological Culture Collection Center (CGMCC) with an accession number of CGMCC No. 25099, dated Jun. 16, 2022. With respect to the chromosome DNA thereof, one or more genes associated with fatty acid metabolism are knocked out or attenuated, and/or a promoter is replaced for enhancement; one or more genes associated with the L-homoserine metabolic pathway are knocked out or attenuated, and/or one or more genes associated with the L-homoserine metabolic pathway are overexpressed or enhanced, and/or one or more genes associated with the L-homoserine metabolic pathway are mutated.

### TECHNICAL FIELD

The present disclosure belongs to the technical field of bioengineering, and particularly relates to an L-homoserine high-yield strain, a construction method therefor, and a use thereof.

### BACKGROUND

L-homoserine is a naturally occurring non-essential amino acid that belongs to the non-protein amino acid family. It serves as a precursor for the synthesis of important high value-added compounds, such as L-methionine. L-homoserine and its derivatives hold significant potential as pharmaceutical intermediates, with a wide range of applications in pharmacology and physiology.

Currently, microbial fermentation methods are widely used both domestically and internationally due to their numerous advantages, including low cost, mild requirements for conditions, and less environmental pollution. In recent years, these methods have become the preferred process for producing various amino acids. However, the microbial fermentation process, which relies solely on glucose as a substrate, faces challenges related to reducing power and energy insufficiency (Glucose+2HCO3−+2NH++2ATP+4NADPH→2HS). In this process, a portion of the glucose is consumed to provide the necessary reducing power, which in turn lowers the sugar-acid conversion efficiency.

Fatty acids have gained increasing attention as a carbon source. Fatty acids possess a higher reducing potential compared to glucose, making them effective not only for providing a carbon backbone but also for delivering substantial reducing power and energy. Taking palmitic acid as an example, the conversion of 1 molecule of palmitic acid to 4 molecules of L-homoserine results in the generation of 11 molecules of FADH and 11 molecules of NADH (C16:0+4NH4+5ATP+8NADPH→4HS+11FADH+11NADH). This process leaves a significant surplus of reducing power and energy after accounting for the necessary 8 molecules of NADPH and 5 molecules of ATP required for the reaction.

In light of this, it is possible to utilize both glucose and palmitic acid as carbon sources for L-homoserine synthesis. By incorporating a small amount of palmitic acid or other fatty acids, reducing power can be effectively provided to maximize the conversion efficiency of glucose into L-homoserine.

### SUMMARY

The primary objective of the present disclosure is to provide an L-homoserine high-yield strain, construction method therefor, and use thereof to address the aforementioned issues. The present disclosure involves the construction of a genetically engineered strain for high L-homoserine yield. By utilizing a dual-substrate system of glucose and fatty acids as carbon sources, the synthesis of L-homoserine is enhanced, resulting in higher yield and improved sugar-acid conversion rates. This approach further reduces production costs, promotes a greener and more environmentally-friendly production process, and offers a distinct competitive advantage in the market.

The objective of the present disclosure and the solutions to its technical problems are achieved through the following technical proposal.

One aspect of the present disclosure provides an L-homoserine high-yield strain of Escherichia coli (strain No.: 13-XA), which is deposited in China General Microbiological Culture Collection Center (CGMCC) with an accession number of CGMCC No. 25099, dated Jun. 16, 2022.

The engineered strain 13-XA is characterized by the knockout or partial weakening of one or more genes related to fatty acid metabolism on the chromosomal DNA, and/or the replacement of promoters to enhance gene expression. One or more genes associated with the L-homoserine metabolic pathway are knocked out or attenuated, and/or one or more genes associated with the L-homoserine metabolic pathway are overexpressed or enhanced, and/or one or more genes associated with the L-homoserine metabolic pathway are mutated. Mutant E. coli is developed by sequentially knocking out the DNA-binding transcriptional dual regulator gene (fadR) and enhancing the promoter of the long-chain fatty acid coenzyme A ligase gene (fadD) in the genome of mutant E. coli ST11. The host strain is then obtained and overexpressed the feedback-relieved aspartate kinase/high-serine dehydrogenase 1 gene, thrA (S345F), resulting in the desired high-yield strain.

The objective of the present disclosure and the solutions to its technical problems are further achieved through the following technical proposal.

Another aspect of the present disclosure provides a method for constructing a highly efficient L-homoserine-producing strain through fermentation, which includes the following steps:

Construction of a host strain: The DNA-binding transcription dual regulator gene (fadR) in the genome of mutant E. coli ST11 is knocked out to obtain mutant E. coli designated as ST12. The promoter of the long-chain fatty acid coenzyme A ligase gene (fadD) is subsequently enhanced, resulting in mutant E. coli designated as ST13.

Construction of a plasmid: The feedback-relieved aspartate kinase/homoserine dehydrogenase 1 gene thrA (S345F) is inserted into the plasmid vector pXB1k between the NcoI and EcoRI sites, generating a recombinant vector designated as pXA.

Construction of an engineered strain: The aforementioned recombinant plasmid pXA is introduced into the mutant E. coli strains ST12 and ST13, respectively, to obtain recombinant engineered strains designated as 12-XA and 13-XA.

The mutant E. coli ST11 is disclosed in Patent 202011270812.X, and its genotype is: E. coli BW25113ΔptsG::glk, ΔgalR::zglf, ΔompT::ppc, ΔldhA::rhtA, ΔlpxM::rhtB, ΔpflB::asd, ΔpoxB::aspA, ΔiclR, ΔlysA, ΔmetA, ΔthrB.

The genotype of the mutant E. coli ST13 is: E. coli ST11ΔfadR, ΔPfadD::PCPA1.

The aspartate kinase/homoserine dehydrogenase 1 gene thrA (S345F) is derived from E. coli K-12 MG1655.

Preferably, the recombinant vector plasmid pXA is constructed as follows:

Using genomic DNA of E. coli K12 as a template, two fragments, thrA-1 and thrA-2, of the feedback-relieved aspartate kinase/homoserine dehydrogenase 1 gene are amplified via PCR with primers thrA-F and S345F-R, and S345F-F and thrA-R. The nucleotide sequence of the forward primer thrA-F is set forth in SEQ ID NO.3, and that of the reverse primer S345F-R is set forth in SEQ ID NO.4. Similarly, the nucleotide sequence of the forward primer S345F-F is set forth in SEQ ID NO.5, and that of the reverse primer thrA-R is set forth in SEQ ID NO.6. The pXB1k vector is digested with the enzymes NcoI and EcoRI, and the resulting large fragment of the vector is recovered. The PCR-amplified thrA-1 and thrA-2 gene fragments are subsequently ligated with the vector's large fragment using the Gibson Assembly method. The ligation products are transformed into competent cells and cultured on LB agar plates containing streptomycin. After overnight incubation at 37° C., monoclonal colonies are selected for plasmid extraction. A pair of primers (pBAD-F and pBAD-R) are designed for PCR verification, and correct clones of the recombinant vector plasmid pXA are screened out. The nucleotide sequence of the forward primer pBAD-F is set forth in SEQ ID NO.7, and that of the reverse primer pBAD-R is set forth in SEQ ID NO.8.

Preferably, the recombinant vector plasmid pXA is obtained by replacing the fragment between the NcoI and EcoRI sites of the pXB1k vector with the feedback-relieved aspartate kinase/homoserine dehydrogenase 1 gene. The nucleotide sequence of the pXB1k vector is set forth in SEQ ID NO. 1, and that of the feedback-relieved aspartate kinase/homoserine dehydrogenase 1 gene is set forth in SEQ ID NO. 2.

Preferably, the mutant E. coli ST13 is constructed as follows:

(1) Using pTargetF as the template, perform PCR amplification with the primer pairs pTarget-fadR-F/pTarget-fadR-R and pTarget-fadDp-F/pTarget-fadDp-R. Digest the amplified fragments with DpnI methylase and transform them into competent E. coli Fast-T1 cells. Screen for positive clones on LB plates containing streptomycin. Verify the positive clones by sequencing using the primer pTarget-cexu-F. Upon confirmation of correct sequencing, designate the constructs as pTarget-fadR and pTarget-fadDp, respectively.

(2) Amplify two separate fragments using PCR with the primer pairs fadR-up500-F/fadR-up500-R and fadR-down500-F/fadR-down500-R. Use the mixture of these two fragments as a template for subsequent PCR amplification with the primer pair fadR-up500-F/fadR-down500-R, yielding the ΔfadR targeting fragment. Amplify three separate fragments using PCR with the primer pairs fadD-up500-F/fadD-up500-R, CPA1-fadD-F/CPA1-fadD-R, and fadD-down500-F/fadD-down500-R. Use the mixture of these three fragments as a template for subsequent PCR amplification with the primer pair fadD-up500-F/fadD-down500-R, yielding the ΔPfadD::PCPA1 targeting fragment. Finally, recover the obtained ΔfadR and ΔPfadD::PCPA1 targeting fragments separately.

(3) Prepare competent cells from the E. coli mutant strain ST11 for transformation. Transform the cells with the pCas plasmid and plate them on LB agar containing kanamycin. Incubate at 30° C. and screen for positive clones.

(4) Select positive clones from step (3) and prepare electrocompetent cells. Mix the electrocompetent cells with the pTarget-fadR plasmid and the ΔfadR targeting fragment. Subject the mixture to electroporation using an electroporation cuvette. Add LB broth medium for recovery at 30° C. and plate the cells on LB agar containing kanamycin and streptomycin. Incubate at 30° C. and screen for positive clones. Verify positive clones by PCR amplification using the primer pair fadR-up700-F/fadR-down700-R and sequence the amplified fragments to confirm successful targeting.

(5) Inoculate the positive clones obtained in the previous step into LB broth medium containing IPTG and kanamycin, and incubate them overnight at 30° C. to eliminate the pTarget-fadR plasmid. After the overnight incubation, streak the culture onto LB agar plates containing kanamycin and incubate them overnight at 30° C. Designate the resulting strain, an E. coli mutant ST11ΔfadR containing the pCas plasmid, as ST12.

(6) Prepare electrocompetent cells from the E. coli mutant ST12 containing the pCas plasmid. Mix these cells with the pTarget-fadDp plasmid and the ΔPfadD::PCPA1 targeting fragment. Repeat steps (4) and (5) to perform the transformation and plasmid elimination procedures. Verify positive clones by sequencing the PCR-amplified fragment using the primer pair fadD-up700-F/fadD-down700-R. Designate the resulting strain, an E. coli mutant ST11ΔfadR, ΔPfadD::PCPA1 containing the pCas plasmid, as ST13.

(7) Inoculate the E. coli mutant strain ST11ΔfadR, ΔPfadD::PCPA1 (ST13), verified by sequencing and containing the pCas plasmid, into LB broth medium and incubate it overnight at 37° C. to eliminate the pCas plasmid. After overnight incubation, streak the culture onto LB agar plates and incubate further overnight at 37° C. Designate the resulting strain, an E. coli mutant ST11ΔfadR, ΔPfadD::PCPA1 free of the pCas plasmid, as ST13.

Preferably, the method also includes the steps for preparing electrocompetent cells: the pCas plasmid is transformed into E. coli ST11 through chemical transformation. Positive clones are selected by incubation at 30° C. on LB plates containing kanamycin. Positive clones are then inoculated into LB broth media containing 2 g/L arabinose and incubated at 30° C. until an OD600 of approximately 0.6 is reached, followed by the preparation of electrocompetent cells.

Preferably, the nucleotide sequence of the forward primer pTarget-fadR-F in step (1) is set forth in SEQ ID NO.9, and that of the reverse primer pTarget-fadR-R is set forth in SEQ ID NO.10. The nucleotide sequence of the forward primer pTarget-fadDp-F is set forth in SEQ ID NO.11, and that of the reverse primer pTarget-fadDp-R is set forth in SEQ ID NO.12.

The PCR amplification system comprises: 10 μL of 5×SF Buffer, 1 μL of dNTP Mix (10 mM each), 20 ng of template pTargetF, 2 μL of each primer (10 μM), 1 μL of Phanta Super-Fidelity DNA Polymerase, and 34 μL of distilled water, with a total volume of 50 μL.

The PCR amplification conditions are as follows: pre-denaturation at 95° C. for 2 min (1 cycle); denaturation at 95° C. for 10 s, annealing at 55° C. for 20 s, extension at 72° C. for 1.5 min (30 cycles); and final extension at 72° C. for 10 min (1 cycle).

Preferably, the nucleotide sequence of the forward primer fadR-up500-F in step (2) is set forth in SEQ ID NO.19, and that of the reverse primer fadR-up500-R is set forth in SEQ ID NO.20. The nucleotide sequence of the forward primer fadR-down500-F is set forth in SEQ ID NO.21, and that of the reverse primer fadR-down500-R is set forth in SEQ ID NO.22. The nucleotide sequence of the forward primer fadD-up500-F is set forth in SEQ ID NO.13, and that of the reverse primer fadD-up500-R is set forth in SEQ ID NO.14. The nucleotide sequence of the forward primer CPA1-fadD-F is set forth in SEQ ID NO.15, and that of the reverse primer CPA1-fadD-R is set forth in SEQ ID NO.16. The nucleotide sequence of the forward primer fadD-down500-F is set forth in SEQ ID NO.17, and that of the reverse primer fadD-down500-R is set forth in SEQ ID NO.18.

The PCR amplification system comprises: 10 μL of 5×SF Buffer, 1 μL of dNTP Mix (10 mM each), 5-20 ng of template, 2 μL of each primer (10 μM), 1 μL of Phanta Super-Fidelity DNA Polymerase, and 34 μL of distilled water, with a total volume of 50 μL.

The PCR amplification conditions are as follows: pre-denaturation at 95° C. for 2 min (1 cycle); denaturation at 95° C. for 10 s, annealing at 55° C. for 20 s, extension at 72° C. for 0.5-2 min (30 s/kb) (30 cycles); and final extension at 72° C. for 10 min (1 cycle).

Preferably, the nucleotide sequence of the forward primer fadR-up700-F in step (3) is set forth in SEQ ID NO.23, and that of the reverse primer fadR-down700-R is set forth in SEQ ID NO.24. The nucleotide sequence of the forward primer fadD-up700-F in step (5) is set forth in SEQ ID NO.25, and that of the reverse primer fadD-down700-R is set forth in SEQ ID NO.26.

The objective of the present disclosure and the solutions to its technical problems are further achieved through the following technical proposal.

Another aspect of the present disclosure provides the application of an L-homoserine high-yield strain for preparing L-homoserine.

Preferably, the application involves inoculating an activated efficient fermentation-producing L-homoserine strain into a fermentation medium and employing a biofermentation process to prepare L-homoserine. The method comprises:

Cultivation at 37° C. with an initial air flow rate of 2 vvm, a stirring speed of 300 rpm, and a dissolved oxygen (DO) concentration set at 100%. During bacterial growth, the air flow rate is adjusted to 3 vvm, and the stirring speed is correlated with the DO value to ensure the DO concentration remained above 30%. After the initial glucose is depleted, replenishment is initiated. Throughout the fermentation, the pH is maintained at 7.0 using ammonia. Once the bacterial density reaches an absorbance (OD600) of 30, L-arabinose with a final concentration of 2 g/L is added to induce protein expression. Following 4 h of induction, palmitic acid with a final concentration of 2 g/L is added, with an additional 2 g/L palmitic acid supplemented every 4 h until the end of fermentation, which concludes when the replenished medium is exhausted.

Preferably, the fermentation medium comprises: citric acid 1-5 g/L, potassium dihydrogen phosphate 1-20 g/L, nitrogen source 1-5 g/L, polyether defoamer 150 μL/L, glucose 5-30 g/L, MgSO4·7H2O 0.3-1 g/L, VB1 5-10 mg/L, lysine 0.1-1 g/L, methionine 0.1-1 g/L, isoleucine 0.1-1 g/L, threonine 0.1-1 g/L, and trace inorganic salt I 1-10 ml/L, with pH 7.0±0.5;

The supplemented medium comprises: glucose 100-800 g/L, MgSO4.7H2O 1-5 g/L, lysine 1-10 g/L, methionine 1-10 g/L, isoleucine 1-10 g/L, threonine 1-10 g/L, palmitic acid 2-5 g/L, and trace inorganic salt II 1-10 mL/L.

Preferably, the trace inorganic salt I in the fermentation medium comprises: EDTA 840 mg/L, CoCl2·6H2O 250 mg/L, MnCl2·4H2O 1500 mg/L, CuCl2·2H2O 150 mg/L, H3BO3300 mg/L, Na2MoO4·2H2O 250 mg/L, Zn(CH3COO)2·2H2O 1300 mg/L, and ferric citrate 10 g/L. The nitrogen source is selected from one or more of ammonium chloride, ammonium acetate, ammonium sulfate, and ammonium phosphate.

The trace inorganic salt II in the supplemented medium comprises: EDTA 1300 mg/L, CoCl2·6H2O 400 mg/L, MnCl2·4H2O 2350 mg/L, CuCl2·2H2O 250 mg/L, H3BO3500 mg/L, Na2MoO4·2H2O 400 mg/L, Zn(CH3COO)2·2H2O 1600 mg/L, and ferric citrate 4 g/L.

Through the above technical solution, the present disclosure offers at least the following advantages: it constructs a mutant E. coli recombinant strain 13-XA capable of efficiently producing L-homoserine.

The present disclosure utilizes genetic engineering to develop an L-homoserine high-yield strain, employing a glucose+palmitic acid culture approach that further reduces production costs. This results in a more environmentally-friendly production process, providing significant competitive advantages in the market. The double-substrate biological production process used in the present disclosure replaces the traditional petrochemical process, utilizing renewable bio-based raw materials instead of non-renewable petrochemical resources. This promotes energy savings, emission reduction, clean production, environmental protection, and the development of a circular economy in the industry. Through continuous optimization and upgrading of strains and manufacturing processes, the resulting product is of higher quality, lower cost, and substantial market potential.

The above description provides an overview of the technical aspects of the present disclosure. To gain a clearer understanding of the technical methods, and for implementation purposes, the following details the preferred embodiments of the present disclosure, along with the drawings.

### BRIEF DESCRIPTION OF THE DRAWINGS

FIG. 1 illustrates the physical map of pXB1k.

FIG. 2 illustrates the curve of L-homoserine production over time during the fermentation process of E. coli 13-XA.

### DETAILED DESCRIPTION OF THE EMBODIMENTS

In order to make the technical means, the creation features, the achievement purposes and the effects of the present disclosure easy to understand, the technical proposals in the embodiments of the present disclosure will be clearly and completely described below with reference to the embodiments and drawings of the present disclosure, and it is obvious that the described embodiments are only a part but not all of the embodiments of the present disclosure. All other embodiments, which can be derived by those skilled in the art from the embodiments given herein without making any creative effort, shall fall within the protection scope of the present disclosure.

In the examples of the present disclosure, the experimental methods used are all conventional methods unless otherwise specified.

The materials and reagents used in the embodiments of the present disclosure are commercially available unless otherwise specified.

The quantitative experiments in the embodiments of the present disclosure are conducted with three repetitions, and the results are averaged.

In the embodiments of the present disclosure, unless otherwise specified, the sequencing validation process is performed by a third-party testing organization, Suzhou GENEWIZ Biotechnology Co., Ltd.

In the embodiments of the present disclosure, E. coli K12 is described in the document “Baba T, Ara T, Hasegawa M, Takai Y, Okumura Y, Baba M, Datsenko K A, Tomita M, Wanner B L, Mori H: Construction of Escherichia coli K-12 in-frame, single-gene knockout mutants: the Keio collection. Mol Syst Biol 2006, 2:2006.0008”. It is a non-pathogenic strain with a well-defined genetic background, short generation time, ease of cultivation, and inexpensive media. The GenBank Accession for the whole genome sequence of E. coli K12 is U00096.3 (GI: 545778205, update date: Aug. 1, 2014, version: 3), which is publicly available from the Institute of Microbiology, Chinese Academy of Sciences. This biological material is solely intended for the replication of experiments related to the present disclosure and may not be used for any other purposes.

One molecule of glucose undergoes glycolysis to generate 2 molecules of phosphoenolpyruvate. Under the action of carboxylase, 2 molecules of CO2 are incorporated to produce 2 molecules of oxaloacetic acid. The oxaloacetic acid is then converted into 2 molecules of aspartate through the action of transaminase (aspC) or via the reverse TCA cycle into 2 molecules of fumaric acid, which is further converted into 2 molecules of aspartate through the action of ammonia lyase (aspA). Aspartate is subsequently converted into aspartate phosphate by the action of bifunctional aspartate kinase. The aspartate phosphate is further transformed into aspartate semialdehyde through aspartate semialdehyde dehydrogenase, which is subsequently converted to 2 molecules of homoserine by the action of bifunctional aspartate kinase. Through the pathway, 1 molecule of glucose can be converted into 2 molecules of homoserine. However, the pathway is limited in reducing power and energy efficiency, necessitating the consumption of a portion of glucose to provide reducing power and energy.

Fatty acids, as a highly reducing carbon source, can also be utilized by the bacterium to produce its necessary nutrients. Additionally, the oxidation of fatty acids provides substantial reducing power. Specifically, it has been calculated that 1 molecule of palmitic acid, when completely oxidized, generates 16 molecules of NADH. In the present disclosure, to enhance the production of homoserine and improve the sugar-acid conversion rate, a small amount of palmitic acid is added during fermentation to supply the necessary reducing power, significantly increasing the yield of homoserine to 154 g/L.

In the embodiments of the present disclosure, the coding sequence for the feedback-relieved aspartate kinase/homoserine dehydrogenase 1 gene is set forth in SEQ ID NO. 2. The coding sequence for the DNA-binding transcriptional dual regulator gene (fadR) is set forth in Gene ID: 948652 (comprising 720 nucleotides), encoding the DNA-binding transcriptional dual regulator set forth in Accession No. NP_415705 (comprising 239 amino acid residues).

The coding sequence for the long-chain fatty acid coenzyme A ligase gene (fadD) is set forth in Gene ID: 946327 (comprising 1686 nucleotides), encoding the long-chain fatty acid coenzyme A ligase set forth in Accession No. NP_416319 (comprising 561 amino acid residues).

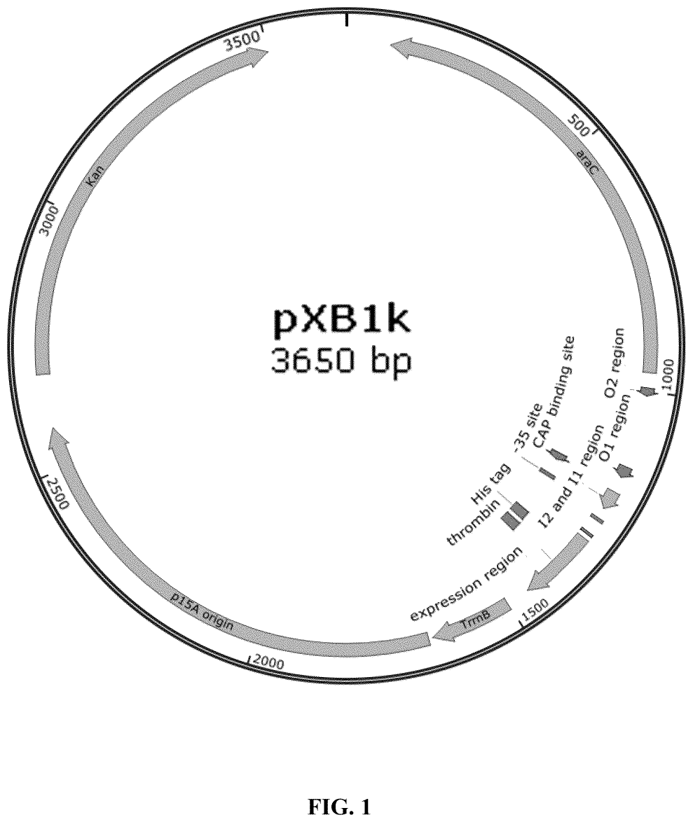

The nucleotide sequence of the pXB1k vector in the following embodiment is set forth in SEQ ID NO.1 and comprises the following fragments: (1) araC-araBAD-MCS fragment (containing arabinose-inducible promoter, multiple cloning site); (2) MCS-TrmB fragment (containing multiple cloning site and TrmB terminator); (3) p15A replication initiation site fragment; (4) Kan fragment of kanamycin resistance gene. The vector map of pXB1k is illustrated in FIG. 1.

SEQ ID NO. 1
aatgtgcctgtcaaatggacgaagcagggattctgcaaaccctat
gctactccgtcaagccgtcaattgtctgattcgttaccaattatg
acaacttgacggctacatcattcactttttcttcacaaccggcac
ggaactcgctcgggctggccccggtgcattttttaaatacccgcg
agaaatagagttgatcgtcaaaaccaacattgcgaccgacggtgg
cgataggcatccgggtggtgctcaaaagcagcttcgcctggctga
tacgttggtcctcgcgccagcttaagacgctaatccctaactgct
ggcggaaaagatgtgacagacgcgacggcgacaagcaaacatgct
gtgcgacgctggcgatatcaaaattgctgtctgccaggtgatcgc
tgatgtactgacaagcctcgcgtacccgattatccatcggtggat
ggagcgactcgttaatcgcttccatgcgccgcagtaacaattgct
caagcagatttatcgccagcagctccgaatagcgcccttcccctt
gcccggcgttaatgatttgcccaaacaggtcgctgaaatgcggct
ggtgcgcttcatccgggcgaaagaaccccgtattggcaaatattg
acggccagttaagccattcatgccagtaggcgcgcggacgaaagt
aaacccactggtgataccattcgcgagcctccggatgacgaccgt
agtgatgaatctctcctggcgggaacagcaaaatatcacccggtc
ggcaaacaaattctcgtccctgatttttcaccaccccctgaccgc
gaatggtgagattgagaatataacctttcattcccagcggtcggt
cgataaaaaaatcgagataaccgttggcctcaatcggcgttaaac
ccgccaccagatgggcattaaacgagtatcccggcagcaggggat
cattttgcgcttcagccatacttttcatactcccgccattcagag
aagaaaccaattgtccatattgcatcagacattgccgtcactgcg
tcttttactggctcttctcgctaaccaaaccggtaaccccgctta
ttaaaagcattctgtaacaaagcgggaccaaagccatgacaaaaa
cgcgtaacaaaagtgtctataatcacggcagaaaagtccacattg
attatttgcacggcgtcacactttgctatgccatagcatttttat
ccataagattagcggatcctacctgacgctttttatcgcaactct
ctactgtttctccatacccgttttttgggctaacaggaggaatta
accatgggtacctctcatcatcatcatcatcacagcagcggcctg
gtgccgcgcggcagcctcgagggtagatctggtactagtggtgaa
ttcggtgagctcggtctgcagctggtgccgcgcggcagccaccac
caccaccaccactaatacagattaaatcagaacgcagaagcggtc
tgataaaacagaatttgcctggcggcagtagcgcggtggtcccac
ctgaccccatgccgaactcagaagtgaaacgccgtagcgccgatg
gtagtgtggggtctccccatgcgagagtagggaactgccaggcat
caaataaaacgaaaggctcagtcgaaagactgggcctttcgtcga
cgcgctagcggagtgtatactggcttactatgttggcactgatga
gggtgtcagtgaagtgcttcatgtggcaggagaaaaaaggctgca
ccggtgcgtcagcagaatatgtgatacaggatatattccgcttcc
tcgctcactgactcgctacgctcggtcgttcgactgcggcgagcg
gaaatggcttacgaacggggcggagatttcctggaagatgccagg
aagatacttaacagggaagtgagagggccgcggcaaagccgtttt
tccataggctccgcccccctgacaagcatcacgaaatctgacgct
caaatcagtggtggcgaaacccgacaggactataaagataccagg
cgtttccccctggcggctccctcgtgcgctctcctgttcctgcct
ttcggtttaccggtgtcattccgctgttatggccgcgtttgtctc
attccacgcctgacactcagttccgggtaggcagttcgctccaag
ctggactgtatgcacgaaccccccgttcagtccgaccgctgcgcc
ttatccggtaactatcgtcttgagtccaacccggaaagacatgca
aaagcaccactggcagcagccactggtaattgatttagaggagtt
agtcttgaagtcatgcgccggttaaggctaaactgaaaggacaag
ttttggtgactgcgctcctccaagccagttacctcggttcaaaga
gttggtagctcagagaaccttcgaaaaaccgccctgcaaggcggt
tttttcgttttcagagcaagagattacgcgcagaccaaaacgatc
tcaagaagatcatcttattaatcagataaaatatttctagatttc
agtgcaatttatctcttcaaatgtagcacctgaagtcagccccat
acgatataagttgtgcggccgccctatttgtttatttttctaaat
acattcaaatatgtatccgctcatgagacaataaccctgataaat
gcttcaataatattgaaaaaggaagagtatgagccatattcaacg
ggaaacgtcttgctctaggccgcgattaaattccaacatggatgc
tgatttatatgggtataaatgggctcgcgataatgtcgggcaatc
aggtgcgacaatctatcgattgtatgggaagcccgatgcgccaga
gttgtttctgaaacatggcaaaggtagcgttgccaatgatgttac
agatgagatggtcagactaaactggctgacggaatttatgcctct
tccgaccatcaagcattttatccgtactcctgatgatgcatggtt
actcaccactgcgatccccgggaaaacagcattccaggtattaga
agaatatcctgattcaggtgaaaatattgttgatgcgctggcagt
gttcctgcgccggttgcattcgattcctgtttgtaattgtccttt
taacagcgaccgcgtatttcgtctcgctcaggcgcaatcacgaat
gaataacggtttggttgatgcgagtgattttgatgacgagcgtaa
tggctggcctgttgaacaagtctggaaagaaatgcataaactttt
gccattctcaccggattcagtcgtcactcatggtgatttctcact
tgataaccttatttttgacgaggggaaattaataggttgtattga
tgttggacgagtcggaatcgcagaccgataccaggatcttgccat
cctatggaactgcctcggtgagttttctccttcattacagaaacg
gctttttcaaaaatatggtattgataatcctgatatgaataaatt
gcagtttcatttgatgctcgatgagtttttctaagaattaattca
tgagcggatacatatttgaatgtatttagaaaaataaacaaatag
gggttccgcgcacatttccccgaaaagtgccacttgcggagaccc
ggtcgtcagcttgtcgtcggttcagggcagggtcgttaaatagcg
catgc
SEQ ID NO. 2
atgcgagtgttgaagttcggcggtacatcagtggcaaatgcagaa
cgttttctgcgtgttgccgatattctggaaagcaatgccaggcag
gggcaggtggccaccgtcctctctgcccccgccaaaatcaccaac
cacctggtggcgatgattgaaaaaaccattagcggccaggatgct
ttacccaatatcagcgatgccgaacgtatttttgccgaacttttg
acgggactcgccgccgcccagccggggttcccgctggcgcaattg
aaaactttcgtcgatcaggaatttgcccaaataaaacatgtcctg
catggcattagtttgttggggcagtgcccggatagcatcaacgct
gcgctgatttgccgtggcgagaaaatgtcgatcgccattatggcc
ggcgtattagaagcgcgcggtcacaacgttactgttatcgatccg
gtcgaaaaactgctggcagtggggcattacctcgaatctaccgtc
gatattgctgagtccacccgccgtattgcggcaagccgcattccg
gctgatcacatggtgctgatggcaggtttcaccgccggtaatgaa
aaaggcgaactggtggtgcttggacgcaacggttccgactactct
gctgcggtgctggctgcctgtttacgcgccgattgttgcgagatt
tggacggacgttgacggggtctatacctgcgacccgcgtcaggtg
cccgatgcgaggttgttgaagtcgatgtcctaccaggaagcgatg
gagctttcctacttcggcgctaaagttcttcacccccgcaccatt
acccccatcgcccagttccagatcccttgcctgattaaaaatacc
ggaaatcctcaagcaccaggtacgctcattggtgccagccgtgat
gaagacgaattaccggtcaagggcatttccaatctgaataacatg
gcaatgttcagcgtttctggtccggggatgaaagggatggtcggc
atggcggcgcgcgtctttgcagcgatgtcacgcgcccgtattttc
gtggtgctgattacgcaatcatcttccgaatacagcatcagtttc
tgcgttccacaaagcgactgtgtgcgagctgaacgggcaatgcag
gaagagttctacctggaactgaaagaaggcttactggagccgctg
gcagtgacggaacggctggccattatctcggtggtaggtgatggt
atgcgcaccttgcgtgggatctcggcgaaattctttgccgcactg
gcccgcgccaatatcaacattgtcgccattgctcagggatcttct
gaacgctcaatctctgtcgtggtaaataacgatgatgcgaccact
ggcgtgcgcgttactcatcagatgctgttcaataccgatcaggtt
atcgaagtgtttgtgattggcgtcggtggcgttggcggtgcgctg
ctggagcaactgaagcgtcagcaaagctggctgaagaataaacat
atcgacttacgtgtctgcggtgttgccaactcgaaggctctgctc
accaatgtacatggccttaatctggaaaactggcaggaagaactg
gcgcaagccaaagagccgtttaatctcgggcgcttaattcgcctc
gtgaaagaatatcatctgctgaacccggtcattgttgactgcact
tccagccaggcagtggcggatcaatatgccgacttcctgcgcgaa
ggtttccacgttgtcacgccgaacaaaaaggccaacacctcgtcg
atggattactaccatcagttgcgttatgcggcggaaaaatcgcgg
cgtaaattcctctatgacaccaacgttggggctggattaccggtt
attgagaacctgcaaaatctgctcaatgcaggtgatgaattgatg
aagttctccggcattctttctggttcgctttcttatatcttcggc
aagttagacgaaggcatgagtttctccgaggcgaccacgctggcg
cgggaaatgggttataccgaaccggacccgcgagatgatctttct
ggtatggatgtggcgcgtaaactattgattctcgctcgtgaaacg
ggacgtgaactggagctggcggatattgaaattgaacctgtgctg
cccgcagagtttaacgccgagggtgatgttgccgcttttatggcg
aatctgtcacaactcgacgatctctttgccgcgcgcgtggcgaag
gcccgtgatgaaggaaaagttttgcgctatgttggcaatattgat
gaagatggcgtctgccgcgtgaagattgccgaagtggatggtaat
gatccgctgttcaaagtgaaaaatggcgaaaacgccctggccttc
tatagccactattatcagccgctgccgttggtactgcgcggatat
ggtgcgggcaatgacgttacagctgccggtgtctttgctgatctg
ctacgtaccctctcatggaagttaggagtctga

### Embodiment 1 Construction of Recombinant Plasmid pXA Expressing Feedback-Relieved Aspartate Kinase/Homoserine Dehydrogenase 1

Using genomic DNA of E. coli K12 as a template, two fragments, thrA-1 and thrA-2, of the feedback-relieved aspartate kinase/homoserine dehydrogenase 1 gene are amplified via PCR with primers thrA-F and S345F-R, and S345F-F and thrA-R. The pXB1k vector is double digested with NcoI and EcoRI, and a large fragment of approximately 3450 bp is recovered. The recovered thrA-1 and thrA-2 gene fragments are ligated to the vector using the Gibson Assembly method (Gibson D G, Young L, Chuang R Y, Venter J C, Hutchison C A, 3rd, Smith H O: Enzymatic assembly of DNA molecules up to several hundred kilobases. Nat Methods 2009, 6:343-345). The ligated product is then transformed into Fast-T1 competent cells (Vazyme Biotech Co., Ltd., catalog C505) and plated on LB agar containing kanamycin. After overnight incubation at 37° C., monoclonal colonies are selected for plasmid extraction. A pair of primers (pBAD-F and pBAD-R) are designed for PCR verification, and correct clones are sent for sequencing. The recombinant vector, designated as pXA, is generated by replacing the fragment between the NcoI and EcoRI sites of the pXB1k vector with the feedback-relieved aspartate kinase/homoserine dehydrogenase 1 gene set forth in SEQ ID NO.2. The primer sequences are as follows:

thrA-F
SEQ ID NO. 3
5′-ggctaacaggaggaattaaccatgcgagtgttgaagttcgg-3′
S345F-R
SEQ ID NO. 4
5′-agcaccacgaaaatacgggcgcgtgacatc-3′
S345F-F
SEQ ID NO. 5
5′-gcccgtattttcgtggtgctgattacgcaatc-3′
thrA-R
SEQ ID NO. 6
5′-gctgcagaccgagctcaccgaattctcagactcctaacttccatg-3′
pBAD-F
SEQ ID NO. 7
5′-cggcgtcacactttgctatg-3′
pBAD-R
SEQ ID NO. 8
5′-cgtttcacttctgagttcggc-3′

In the feedback-relieved aspartate kinase/homoserine dehydrogenase 1 gene expression cassette, the promoter responsible for initiating transcription of the feedback-relieved aspartate kinase/homoserine dehydrogenase 1 gene is the pBAD promoter.

### Embodiment 2 Construction of E. coli Mutant ST11

The E. coli mutant ST11 described in this embodiment is constructed according to the method outlined in the Chinese patent application CN202011270812.X.

Embodiment 3 Construction of Escherichia coli mutant ST13

The E. coli mutant ST13 is constructed by utilizing CRISPR technology (Jiang Y, Chen B, Duan C, Sun B, Yang J, Yang S: Multigene editing in the Escherichia coli genome via the CRISPR-Cas9 system. Appl Environ Microbiol 2015, 81:2506-2514.) to knock out the DNA-binding transcriptional repressor gene (fadR) of E. coli ST11 and enhance the promoter of the long-chain fatty acid coenzyme A ligase gene (fadD), resulting in the E. coli mutant ST11ΔfadR CPA1-fadD, designated as ST13 in this application.

Specifically, in this embodiment, the E. coli mutant ST13 is obtained by knocking out the DNA binding transcription repressor gene (fadR) of E. coli ST11 and enhancing the promoter of the long-chain fatty acid coenzyme A ligase gene (fadD) (designated as ST13). The specific steps for constructing E. coli mutant ST11 are as follows:

(1) Preparation of electrocompetent cells: The pCas plasmid (Jiang Y, Chen B, Duan C, Sun B, Yang J, Yang S: Multigene editing in the Escherichia coli genome via the CRISPR-Cas9 system. Appl Environ Microbiol 2015, 81:2506-2514.) is transformed into E. coli ST11 through chemical transformation. Positive clones are selected by culturing on LB plates containing 50 μg/mL kanamycin at 30° C. Positive clones are then inoculated into LB broth media containing 2 g/L arabinose and cultured at 30° C. until the optical density at 600 nm (OD600) reaches approximately 0.6, followed by the preparation of electrocompetent cells.

(2) Construction of pTarget plasmid: The website https://crispy.secondarymetabolites.org is utilized to select the N20 of the knockdown site, and primers are designed to construct the pTarget plasmid. Using pTargetF (Jiang Y, Chen B, Duan C, Sun B, Yang J, Yang S: Multigene editing in the Escherichia coli genome via the CRISPR-Cas9 system. Appl Environ Microbiol 2015, 81:2506-2514.) as a template, PCR amplification is performed with primers pTarget-fadDp-F and pTarget-fadDp-R, and pTarget-fadR-F and pTarget-fadR-R, respectively, resulting in fragments approximately 2100 bp in size.

The PCR amplification system comprises: 10 μL of 5×SF Buffer, 1 μL of dNTP Mix (10 mM each), 20 ng of template pTargetF, 2 μL of each primer (10 μM), 1 μL of Phanta Super-Fidelity DNA Polymerase (Vazyme Biotech Co., Ltd., product catalog P501), and 34 μL of distilled water, with a total volume of 50 μL. The amplification conditions are as follows: initial denaturation at 95° C. for 2 min (1 cycle); denaturation at 95° C. for 10 s, annealing at 55° C. for 20 s, extension at 72° C. for 1.5 min (30 cycles); and final extension at 72° C. for 10 min (1 cycle). After incubating with DpnI methylase for approximately 3 h, the E. coli Fast-T1 competent cells are directly transformed using the chemical transformation method. Positive clones are screened on LB plates containing streptomycin (concentration of 50 μg/mL) and verified by sequencing with primer pTarget-cexu-F. Following successful sequencing, they are designated as pTarget-fadD and pTarget-fadR, respectively. The sequences of the primers are as follows (with the N20 sequence underlined):

pTarget-fadDp-F
SEQ ID NO. 9
5′-ACGACGAACACGCATTTTAGGTTTTAGAGCTAGAAATAGC-3′
pTarget-fadDp-R
SEQ ID NO. 10
5′-CTAAAATGCGTGTTCGTCGTACTAGTATTATACCTAGGAC-3′
pTarget-fadR-F
SEQ ID NO. 11
5′-GCTGGCTACCGCTAATGAAGgttttagagctagaaatagc-3′
pTarget-fadR-R
SEQ ID NO. 12
5′-cttcattagcggtagccagcactagtattatacctaggac-3′

(3) Amplification of the targeting fragments: PCR amplification is performed using primer pairs fadD-up500-F/fadD-up500-R, CPA1-fadD-F/CPA1-fadD-R, and fadD-down500-F/fadD-down500-R, resulting in fragments of approximately 500 bp, 1700 bp, and 500 bp, respectively. Using a mixture of these three fragments as a template, PCR amplification with primers fadD-up500-F and fadD-down500-R yields a targeting fragment, fadD::CPA1-fadD, of approximately 2700 bp in size.

Similarly, PCR amplification with fadR-up500-F/fadR-down500-R and fadR-down500-F/fadR-down500-R yields fragments of approximately 500 bp each. Using a mixture of these two fragments as a template, PCR amplification with primers fadR-up500-F and fadR-down500-R produces a ΔfadR targeting fragment of approximately 1000 bp in size.

The PCR amplification system comprises: 10 μL of 5×SF Buffer, 1 μL of dNTP Mix (10 mM each), 5-20 ng of template, 2 μL of each primer (10 μM), 1 μL of Phanta Super-Fidelity DNA Polymerase (Vazyme Biotech Co., Ltd., product catalog P501), and 34 μL of distilled water, with a total volume of 50 μl. The amplification conditions are as follows: initial denaturation at 95° C. for 2 min (1 cycle); denaturation at 95° C. for 10 s, annealing at 55° C. for 20 s, extension at 72° C. for 0.5-2 min (30 s/kb) (30 cycles); and final extension at 72° C. for 10 min (1 cycle).

The targeting fragments, fadD::CPA1-fadD and ΔfadR, are recovered separately. Each fragment comprises a 500 bp upstream homology arm, a replacement gene expression cassette, and a 500 bp downstream homology arm, in that order. The primer sequences used are as follows:

fadD-up500-F
SEQ ID NO. 13
5′-attaaaggcagcagtcccac-3′
fadD-up500-R
SEQ ID NO. 14
5′-TATAAGGAGGgctgttttttttctttaaaaac-3′
CPA1-fadD-F
SEQ ID NO. 15
5′-aagaaacagcCCTCCTTATAACTTCGTATAATG
CPA1-fadD-R
SEQ ID NO. 16
5′-ccttcttcatGATATCTCCTTCGTAAAAGATC-3′
fadD-down500-F
SEQ ID NO. 17
5′-AGGAGATATCatgaagaaggtttggcttaac-3′
fadD-down500-R
SEQ ID NO. 18
5′-tcggcaccaaacgcttgatg-3′
fadR-up500-F
SEQ ID NO. 19
5′-acttcaagatttgccgccac-3′
fadR-up500-R
SEQ ID NO. 20
5′-gaatggctaacatagtgagatttccataacac-3′
fadR-down500-F
SEQ ID NO. 21
5′-tctcactatgttagccattcaggggcgata-3′
fadR-down500-R
SEQ ID NO. 22
5′-gatatcgccggttccgactg-3′

(4) Electroporation: A mixture of 200 ng of pTarget-fadR plasmid, 400 ng of ΔfadR targeting fragment, and 100 μL of electrocompetent cells prepared in step (1) is prepared. The mixture is transferred into a 2 mm electroporation cuvette, and a 2.5 kV pulse is applied. Then, 1 mL of LB broth medium is added, and the mixture is incubated at 30° C. for recovery. Subsequently, the cells are spread on LB plates containing kanamycin and streptomycin (kanamycin concentration of 50 μg/ml, streptomycin concentration of 50 μg/ml) and incubated at 30° C. Positive clones are then screened. PCR amplification is performed using primers fadD-up700-F and fadD-down700-R, and the amplified fragments are verified by sequencing. The PCR amplification system comprises: 10 μL of Green Taq Mix (Vazyme Biotech Co., Ltd., product catalog P131), 0.8 μL of each primer (10 μM), 8.4 μL of distilled water, and 0.2 μL of template bacterial solution, with a total volume of 20 μL. The PCR amplification conditions are as follows: pre-denaturation at 95° C. for 3 min (1 cycle); denaturation at 95° C. for 15 s, annealing at 55° C. for 15 s, extension at 72° C. for 1-5 min (60 s/kb) (30 cycles); and final extension at 72° C. for 5 min (1 cycle).

(5) Elimination of pTarget plasmid: Positive clones verified by sequencing are inoculated in LB broth medium containing 0.1 mM IPTG and kanamycin, and incubated at 30° C. overnight to facilitate the elimination of the pTarget plasmid. After overnight incubation, the strain is streaked onto LB agar plates containing kanamycin and incubated at 30° C. overnight. This results in E. coli mutant ST11ΔfadR containing the pCas plasmid, designated as ST12.

(6) Monoclonal colonies are picked from the plate in step (5), and electrocompetent cells are prepared. These cells are then mixed with the pTarget-fadDp plasmid and fadD::CPA1-fadD targeting fragment, and the steps in (4)-(5) are repeated. Sequencing is performed using primers fadD-up700-F and fadD-down700-R for verification. This process results in the generation of E. coli mutant ST11ΔfadR CPA1-fadD, designated as ST13, which contains the pCas plasmid.

(7) Elimination of pCas plasmid: The E. coli mutant ST11ΔfadR CPA1-fadD (ST13), containing the pCas plasmid, is inoculated into LB broth media and incubated at 37° C. overnight to eliminate the pCas plasmid. The overnight culture is streaked onto LB agar plates and incubated at 37° C. overnight to obtain plasmid-free E. coli mutant ST11ΔfadR CPA1-fadD (ST13).

The primer sequences used for verification and sequencing are as follows:

fadR-up700-F
SEQ ID NO. 23
5′-tgtcttcggtacgggaagag-3′
fadR-down700-R
SEQ ID NO. 24
5′-ggcactacaccatccttaac-3′
fadD-up700-F
SEQ ID NO. 25
5′-taaaacggtggcggtggaac-3′
fadD-down700-R
SEQ ID NO. 26
5′-gtcgcgttaacctgttccag-3′

### Embodiment 4 Construction of Engineered Strain 13-XA for High L-Homoserine Yield

The expression vector pXA, constructed in Embodiment 1, is transformed into E. coli mutant ST13 through chemical transformation. Positive clones are screened on LB plates containing kanamycin (kanamycin concentration: 50 μg/ml), and the resulting clone strain is designated as 13-XA.

### Embodiment 5 High-Density Fermentation of Strain 13-XA

The fermentation medium comprises: citric acid 1.7 g/L, potassium dihydrogen phosphate 14 g/L, diammonium hydrogen phosphate 4 g/L, polyether defoamer 150 μl/L, glucose 20 g/L, MgSO4·7H2O 0.6 g/L, VB1 9 mg/L, lysine 0.4 g/L, methionine 0.2 g/L, isoleucine 0.2 g/L, threonine 0.3 g/L, and trace inorganic salt I 10 mL/L, with pH 7.0. Trace inorganic salt I comprises: EDTA 840 mg/L, CoCl2·6H2O 250 mg/L, MnCl2·4H2O 1500 mg/L, CuCl2·2H2O 150 mg/L, H3BO3300 mg/L, Na2MoO4·2H2O 250 mg/L, Zn(CH3COO)2·2H2O 1300 mg/L, and ferric citrate 10 g/L. The fed-batch medium comprises: glucose 600 g/L, MgSO4·7H2O 2 g/L, lysine 4 g/L, methionine 2 g/L, isoleucine 2 g/L, threonine 3 g/L, palmitic acid 5 g/L, and trace inorganic salt II 10 ml/L. Trace inorganic salt II comprises: EDTA 1300 mg/L, CoCl2·6H2O 400 mg/L, MnCl2·4H2O 2350 mg/L, CuCl2·2H2O 250 mg/L, H3BO3500 mg/L, Na2MoO4·2H2O 400 mg/L, Zn(CH3COO)2·2H2O 1600 mg/L, and ferric citrate 4 g/L. Add 2 g/L of fatty acid after 4 h of induction, and replenish 2 g/L every 4 h. Technicians in the field may adjust the composition of the above components within a reasonable range according to specific conditions. This embodiment presents only one specific embodiment. As an alternative to this embodiment, the composition of the fermentation medium may be adjusted within the following ranges: citric acid 1-5 g/L, potassium dihydrogen phosphate 1-20 g/L, nitrogen source 1-5 g/L, glucose 5-30 g/L, MgSO4·7H2O 0.3-1 g/L, VB1 5-10 mg/L, lysine 0.1-1 g/L, methionine 0.1-1 g/L, isoleucine 0.1-1 g/L, threonine 0.1-1 g/L, and trace inorganic salt I 1-10 mL/L, with pH 7.0±0.5.

The nitrogen source is an inorganic nitrogenous compound, which may be selected from one or more of the ammonium chloride, ammonium acetate, ammonium sulfate, and ammonium phosphate. The trace inorganic salt is selected from one or more of the soluble iron, cobalt, copper, zinc, manganese, and molybdate salts.

The composition of the fed-batch medium may be adjusted within the following ranges: glucose 100-800 g/L, MgSO4·7H2O 1-5 g/L, lysine 1-10 g/L, methionine 1-10 g/L, isoleucine 1-10 g/L, threonine 1-10 g/L, palmitic acid 2-5 g/L, and trace inorganic salt II 1-10 mL/L.

The fatty acid is selected from one or more of the palmitic acid, oleic acid, lauric acid, and soybean oil, with the amounts and timing of addition adjusted based on experience.

Seed culture: 100 mL of LB medium is prepared in a 250 mL triangular flask and sterilized at 121° C. for 20 min. After cooling, glycerol-preserved strain 13-A stored at −80° C. is inoculated. The culture is maintained at 37° C. with shaking at 200 rpm for 6-8 h and used for inoculating the fermentation medium. Technicians in the field may adjust these conditions within a reasonable range according to specific requirements, without affecting the intended purpose of the present disclosure. This embodiment presents only one specific embodiment. As an alternative, the culture conditions may be adjusted within the following ranges: culture temperature ranging from 25 to 42° C. and shaker speed ranging from 100 to 300 rpm.

Fermenter Inoculation: As a preferred embodiment, the fermentation medium volume in a 5 L fermenter is 2.5 L. Following sterilization, the seed liquid is inoculated with a volume of 5% (V/V). The initial glucose concentration is set at 20 g/L. The fermentation is maintained at 37° C. with an initial air flow rate of 2 vvm, a stirring speed of 300 rpm, and a dissolved oxygen concentration set at 100%. During bacterial growth, the air flow is adjusted to 3 vvm, and stirring speed is correlated with the DO value to ensure the dissolved oxygen concentration remained above 30%. Once the initial glucose is depleted, replenishment is initiated. pH is controlled at 7.0 using ammonia throughout the fermentation process. Once the bacterial density reaches an absorbance (OD600) of 30, L-arabinose with a final concentration of 2 g/L is added to induce protein expression. Following 4 h of induction, palmitic acid with a final concentration of 2 g/L is added, with an additional 2 g/L palmitic acid supplemented every 4 h until the end of fermentation, which concludes when the replenished medium is exhausted. Technicians in the field may adjust these conditions within a reasonable range according to specific requirements, without affecting the intended purpose of the present disclosure.

Analytical method: Components in the fermentation broth are determined using an Agilent (Agilent-1200) high-performance liquid chromatography (HPLC) system. The method for determining L-homoserine is as follows: The sample is appropriately diluted and derivatized with 2,4-dinitrofluorobenzene (DNFB). A 100 μL sample is combined with 50 μL of 10 g/L DNFB acetonitrile solution and 100 μL of 0.5 M NaHCO3 solution, thoroughly mixed, and reacts at 60° C. in the dark for 1 h. After cooling, 750 μL of 0.01 M KH₂PO₄ solution is added, and the mixture is further homogenized. The solution is filtered through a 0.22 μm membrane, followed by HPLC detection. The chromatographic separation is conducted using a ZORBAX Eclipse XDB-C18 column (4.6×150 mm, 5 μm; Agilent) at 30° C. The mobile phase comprises 35% acetonitrile-formic acid (0.1%) aqueous solution, with a flow rate of 1 mL/min, and the detection wavelength is set at 360 nm.

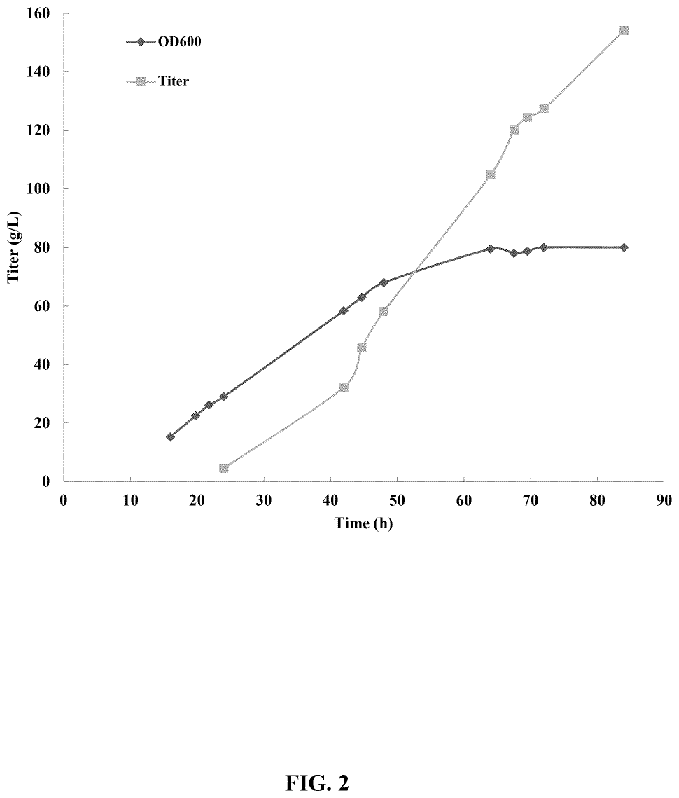

Results: As illustrated in FIG. 2, the yield of L-homoserine in the conversion solution reaches 154 g/L, and the conversion rate of L-homoserine during the entire fermentation stage is up to 0.65 g L-homoserine/g glucose.

### Comparative Embodiment 1

Patent No. CN201710953111.8 describes E. coli K-12 MG1655 strain with modifications including the knockout of the thrB gene, overexpression of the rhtA gene, knockout of the thrL gene, mutation of the thrA gene, and the multicopy expression of thrA*, ppc, aspA, pntA and pntB on chromosomal DNA (MG1655 Δ thrB, rhtA 23, Δ thrL, thrA* (G433R), ΔcadA::thrA*-ppc-aspA-pntAB, ΔyidJ::thrA*-ppc-aspA-pntAB, ΔatpC::thrA*-ppc-aspA-pntAB, ΔdacA::thrA*, ΔbcsB::thrA*, ΔmenH::aspC, ΔyddB::asd), yielding an engineered strain Hom8 with high L-homoserine production, capable of producing 88.1 g/L of L-homoserine by fermentation, which is significantly lower than the yield of the strain described in the present disclosure.

The above is only a preferred embodiment of the present disclosure, and is not intended to limit the scope of the present disclosure. Although the present disclosure has been disclosed in the above preferred embodiments, it is not intended to limit the present disclosure. Those skilled in the art can make some modifications or modifications to the equivalent embodiments by using the above-disclosed technical contents without departing from the technical scope of the present disclosure, but without departing from the technical solution of the present disclosure, according to the present disclosure. Any modification, equivalent change and modification of the above embodiments according to the technical substantials of the present disclosure are still within the scope of the technical solution of the present disclosure.

What is claimed is:

1. An L-homoserine high-yield strain, wherein the L-homoserine high-yield strain is Escherichia coli with a strain No. of 13-XA and is deposited in China General Microbiological Culture Collection Center (CGMCC) with an accession number of CGMCC No. 25099, dated Jun. 16, 2022.

2. A method for constructing the L-homoserine high-yield strain according to claim 1, comprising the following steps:
constructing a host strain, comprising: knocking out the DNA-binding transcription dual regulator gene (fadR) in the genome of mutant E. coli ST11 to obtain a mutant strain designated as E. coli ST12, and enhancing the promoter of the long-chain fatty acid coenzyme A ligase gene (fadD) in the genome of mutant E. coli ST12, resulting in a mutant strain designated as E. coli ST13;
constructing a plasmid, comprising: inserting the feedback-relieved aspartate kinase/homoserine dehydrogenase 1 gene thrA (S345F) into the plasmid vector pXB1k between the NcoI and EcoRI restriction sites to generate a recombinant plasmid designated as pXA;
constructing an engineered strain, comprising: introducing the recombinant plasmid pXA into the mutant strains E. coli ST12 and E. coli ST13, respectively, to obtain recombinant engineered strains designated as 12-XA and 13-XA,
wherein the mutant E. coli ST11 is disclosed in Patent 202011270812.X, and its genotype is: E. coli BW25113ΔptsG::glk, ΔgalR::zglf, ΔompT::ppc, ΔldhA::rhtA, ΔlpxM::rhtB, ΔpflB::asd, ΔpoxB::aspA, ΔiclR, ΔlysA, ΔmetA, ≢thrB;
the genotype of the mutant E. coli ST13 is: E. coli ST11ΔfadR, ΔP_{fadD}::P_{CPA1}; and
the aspartate kinase/homoserine dehydrogenase 1 gene thrA (S345F) is derived from E. coli K-12 MG1655.

3. A method for constructing the L-homoserine high-yield strain according to claim 2, wherein the recombinant vector plasmid pXA is constructed by: amplifying two fragments, thrA-1 and thrA-2, of the feedback-relieved aspartate kinase/homoserine dehydrogenase 1 gene from the genomic DNA of E. coli K12 using PCR and primers thrA-F and S345F-R, and S345F-F and thrA-R, wherein the nucleotide sequence of the forward primer thrA-F is set forth in SEQ ID NO.3, the reverse primer S345F-R is set forth in SEQ ID NO.4, the forward primer S345F-F is set forth in SEQ ID NO.5, and the reverse primer thrA-R is set forth in SEQ ID NO.6; digesting the plasmid vector pXB1k with restriction enzymes NcoI and EcoRI to generate a large vector fragment; ligating the PCR-amplified thrA-1 and thrA-2 fragments with the large vector fragment using the Gibson Assembly method to produce ligation products; transforming the ligation products into competent cells, plating the transformed cells on LB agar plates containing streptomycin, incubating the plates at 37° C. overnight, and selecting monoclonal colonies for plasmid extraction; verifying the recombinant plasmid by PCR using primers pBAD-F and pBAD-R, wherein the nucleotide sequence of the forward primer pBAD-F is set forth in SEQ ID NO.7, and the reverse primer pBAD-R is set forth in SEQ ID NO.8; and screening for correct clones of the recombinant vector plasmid pXA.

4. A method for constructing the L-homoserine high-yield strain according to claim 3, comprising the following steps: obtaining the recombinant vector plasmid pXA by replacing the fragment between the NcoI and EcoRI sites of the pXB1k vector with the feedback-relieved aspartate kinase/homoserine dehydrogenase 1 gene (thrA), wherein the nucleotide sequence of the pXB1k vector is set forth in SEQ ID NO.1 and the nucleotide sequence of the feedback-relieved aspartate kinase/homoserine dehydrogenase 1 gene is set forth in SEQ ID NO.2.

5. A method for constructing the L-homoserine high-yield strain according to claim 2, wherein the mutant E. coli strain ST13 is constructed by:
(1) performing PCR amplification using the plasmid pTargetF as a template and primer pairs pTarget-fadR-F/pTarget-fadR-R and pTarget-fadDp-F/pTarget-fadDp-R, digesting the amplified fragments with DpnI methylase, transforming the fragments into competent E. coli Fast-TI cells, screening for positive clones on LB plates containing streptomycin, verifying the positive clones by sequencing with the primer pTarget-cexu-F, and designating the resulting constructs as pTarget-fadR and pTarget-fadDp, respectively;
(2) amplifying a ΔfadR targeting fragment by performing PCR amplification with primer pairs fadR-up500-F/fadR-up500-R and fadR-down500-F/fadR-down500-R to generate two fragments, using the mixture of the two fragments as a template for PCR amplification with the primer pair fadR-up500-F/fadR-down500-R, amplifying a ΔP_{fadD}::P_{CPA1} targeting fragment by performing PCR amplification with primer pairs fadD-up500-F/fadD-up500-R, CPA1-fadD-F/CPA1-fadD-R, and fadD-down500-F/fadD-down500-R to generate three fragments, using the mixture of the three fragments as a template for PCR amplification with the primer pair fadD-up500-F/fadD-down500-R, and recovering the obtained ΔfadR and ΔP_{fadD}::P_{CPA1} targeting fragments separately;
(3) preparing competent cells from the E. coli mutant strain ST11, transforming the cells with the pCas plasmid, plating the transformed cells on LB agar containing kanamycin, incubating the plates at 30° C., and screening for positive clones;
(4) preparing electrocompetent cells from positive clones obtained in step (3), mixing the cells with the pTarget-fadR plasmid and the ΔfadR targeting fragment, performing electroporation, recovering the cells in LB broth medium at 30° C., plating the cells on LB agar containing kanamycin and streptomycin, incubating the plates at 30° C., screening for positive clones, verifying the positive clones by PCR amplification with the primer pair fadR-up700-F/fadR-down700-R, and sequencing the amplified fragments to confirm successful targeting;
(5) incubating the positive clones obtained in step (4) in LB broth medium containing IPTG and kanamycin overnight at 30° C. to eliminate the pTarget-fadR plasmid, streaking the culture onto LB agar plates containing kanamycin, incubating the plates overnight at 30° C., and designating the resulting strain as E. coli mutant ST11ΔfadR containing the pCas plasmid (ST12);
(6) preparing electrocompetent cells from the E. coli mutant strain ST12, mixing the cells with the pTarget-fadDp plasmid and the ΔP_{fadD}::P_{CPA1} targeting fragment, performing the steps of transformation, plasmid elimination, and screening as in steps (4) and (5), verifying positive clones by sequencing the PCR-amplified fragment with the primer pair fadD-up700-F/fadD-down700-R, and designating the resulting strain as E. coli mutant ST11ΔfadR, ΔP_{fadD}::P_{CPA1} containing the pCas plasmid (ST13); and
(7) incubating the E. coli mutant strain ST13 verified by sequencing and containing the pCas plasmid in LB broth medium overnight at 37° C. to eliminate the pCas plasmid, streaking the culture onto LB agar plates, incubating the plates overnight at 37° C., and designating the resulting strain as E. coli mutant ST11ΔfadR, ΔP_{fadD}::P_{CPA1} free of the pCas plasmid (ST13).

6. A method for constructing the L-homoserine high-yield strain according to claim 5, comprising the following steps: preparing electrocompetent cells by introducing the pCas plasmid into E. coli ST11 through chemical transformation, screening positive clones on LB agar plates containing kanamycin at 30° C., inoculating the selected positive clones into LB broth medium containing 2 g/L arabinose, culturing the clones at 30° C. until the optical density at 600 nm (OD₆₀₀) reaches approximately 0.6, and preparing electrocompetent cells from the cultured clones.

7. A method for constructing the L-homoserine high-yield strain according to claim 5, comprising the following steps: defining the nucleotide sequence of the forward primer pTarget-fadR-F as set forth in SEQ ID NO.9, the reverse primer pTarget-fadR-R as set forth in SEQ ID NO.10, the forward primer pTarget-fadDp-Fas set forth in SEQ ID NO.11, and the reverse primer pTarget-fadDp-R as set forth in SEQ ID NO.12; performing PCR amplification using a system comprising 10 μL of 5×SF Buffer, 1 μL of dNTP Mix (10 mM each), 20 ng of template pTargetF, 2 μL of each primer (10 μM), 1 μL of Phanta Super-Fidelity DNA Polymerase, and 34 μL of distilled water to a total volume of 50 μL; and conducting PCR amplification under the following conditions: pre-denaturation at 95° C. for 2 min (1 cycle), denaturation at 95° C. for 10 s, annealing at 55° C. for 20 s, extension at 72° C. for 1.5 min (30 cycles), and final extension at 72° C. for 10 min (1 cycle).

8. A method for constructing the L-homoserine high-yield strain according to claim 5, comprising the following steps: defining the nucleotide sequence of the forward primer fadR-up500-F in step (2) as set forth in SEQ ID NO.19, the reverse primer fadR-up500-R as set forth in SEQ ID NO.20, the forward primer fadR-down500-F as set forth in SEQ ID NO.21, the reverse primer fadR-down500-R as set forth in SEQ ID NO.22, the forward primer fadD-up500-F as set forth in SEQ ID NO.13, the reverse primer fadD-up500-R as set forth in SEQ ID NO.14, the forward primer CPA1-fadD-F as set forth in SEQ ID NO.15, the reverse primer CPA1-fadD-R as set forth in SEQ ID NO.16, the forward primer fadD-down500-F as set forth in SEQ ID NO.17, and the reverse primer fadD-down500-R as set forth in SEQ ID NO.18; performing PCR amplification using a system comprising 10 μL of 5×SF Buffer, 1 μL of dNTP Mix (10 mM each), 5-20 ng of template, 2 μL of each primer (10 μM), 1 μL of Phanta Super-Fidelity DNA Polymerase, and 34 μL of distilled water to a total volume of 50 L; and conducting PCR amplification under the following conditions: pre-denaturation at 95° C. for 2 min (1 cycle), denaturation at 95° C. for 10 s, annealing at 55° C. for 20 s, extension at 72° C. for 0.5-2 min (30 s/kb) for 30 cycles, and a final extension at 72° C. for 10 min (1 cycle).

9. A method for constructing the L-homoserine high-yield strain according to claim 5, comprising the following steps: defining the nucleotide sequence of the forward primer fadR-up700-F in step (3) as set forth in SEQ ID NO.23, the reverse primer fadR-down700-R in step (3) as set forth in SEQ ID NO.24, the forward primer fadD-up700-F in step (5) as set forth in SEQ ID NO.25, and the reverse primer fadD-down700-R in step (5) as set forth in SEQ ID NO.26.

10. A use of the L-homoserine high-yield strain according to claim 1, employing the L-homoserine high-yield strain for preparing L-homoserine.

11. A use of the L-homoserine high-yield strain according to claim 10, employing a biofermentation process to prepare L-homoserine, wherein the method comprises:
inoculating an activated, highly efficient L-homoserine-producing strain into a fermentation medium and cultivating the strain at 37° C. with an initial air flow rate of 2 vvm, a stirring speed of 300 rpm, and a dissolved oxygen (DO) concentration set at 100%; adjusting the air flow rate to 3 vvm and correlating the stirring speed with the DO value during bacterial growth to maintain the DO concentration above 30%; initiating glucose replenishment after the initial glucose is depleted and maintaining the pH at 7.0 using ammonia; adding L-arabinose at a final concentration of 2 g/L to induce protein expression once the bacterial density reaches an optical density (OD₆₀₀) of 30;
adding palmitic acid at a final concentration of 2 g/L after 4 h of induction and supplementing an additional 2 g/L of palmitic acid every 4 h until the end of fermentation, which concludes upon exhaustion of the replenished medium.

12. A use of the L-homoserine high-yield strain according to claim 10, wherein the fermentation medium comprises: citric acid at 1-5 g/L, potassium dihydrogen phosphate at 1-20 g/L, a nitrogen source at 1-5 g/L, polyether defoamer at 150 L/L, glucose at 5-30 g/L, MgSO₄·7H₂O at 0.3-1 g/L, vitamin B1 (VB1) at 5-10 mg/L, lysine at 0.1-1 g/L, methionine at 0.1-1 g/L, isoleucine at 0.1-1 g/L, threonine at 0.1-1 g/L, and trace inorganic salt I at 1-10 mL/L, with a pH of 7.0+0.5, and wherein the supplemented medium comprises glucose at 100-800 g/L, MgSO₄·7H₂O at 1-5 g/L, lysine at 1-10 g/L, methionine at 1-10 g/L, isoleucine at 1-10 g/L, threonine at 1-10 g/L, palmitic acid at 2-5 g/L, and trace inorganic salt II at 1-10 mL/L.

13. A use of the L-homoserine high-yield strain according to claim 12, wherein: the trace inorganic salt I in the fermentation medium comprises EDTA at 840 mg/L, CoCl₂·6H₂O at 250 mg/L, MnCl₂·4H₂O at 1500 mg/L, CuCl₂·2H₂O at 150 mg/L, H₃BO₃ at 300 mg/L, Na₂MoO₄·2H₂O at 250 mg/L, Zn(CH₃COO)₂·2H₂O at 1300 mg/L, and ferric citrate at 10 g/L, and the nitrogen source is selected from one or more of ammonium chloride, ammonium acetate, ammonium sulfate, and ammonium phosphate;
the trace inorganic salt II in the supplemented medium comprises EDTA at 1300 mg/L, CoCl₂·6H₂O at 400 mg/L, MnCl₂·4H₂O at 2350 mg/L, CuCl₂·2H₂O at 250 mg/L, H₃BO₃ at 500 mg/L, Na₂MoO₄·2H₂O at 400 mg/L, Zn(CH₃COO)₂·2H₂O at 1600 mg/L, and ferric citrate at 4 g/L.


SEQUENCE LISTING (26 sequences)

SEQ ID NO: 1 - DNA, 3650 bp, synthetic construct
  source 1..3650: mol_type = other DNA; note = Nucleotide sequence of pXB1k vector
aatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgggtacctctcatcatcatcatcatcacagcagcggcctggtgccgcgcggcagcctcgagggtagatctggtactagtggtgaattcggtgagctcggtctgcagctggtgccgcgcggcagccaccaccaccaccaccactaatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgtcgacgcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgcggccgccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgaccgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacttgcggagacccggtcgtcagcttgtcgtcggttcagggcagggtcgttaaatagcgcatgc

SEQ ID NO: 2 - DNA, 2463 bp, Escherichia coli
  source 1..2463: mol_type = genomic DNA; note = Nucleotide sequence of feedback-relieved aspartate kinase/homoserine dehydrogenase 1 gene
atgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtattttcgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctga

SEQ ID NO: 3 - DNA, 41 bp, synthetic construct
  source 1..41: mol_type = other DNA; note = Forward primer thrA-F
ggctaacaggaggaattaaccatgcgagtgttgaagttcgg

SEQ ID NO: 4 - DNA, 30 bp, synthetic construct
  source 1..30: mol_type = other DNA; note = Reverse primer S345F-R
agcaccacgaaaatacgggcgcgtgacatc

SEQ ID NO: 5 - DNA, 32 bp, synthetic construct
  source 1..32: mol_type = other DNA; note = Forward primer S345F -F
gcccgtattttcgtggtgctgattacgcaatc

SEQ ID NO: 6 - DNA, 45 bp, synthetic construct
  source 1..45: mol_type = other DNA; note = Reverse primer thrA-R
gctgcagaccgagctcaccgaattctcagactcctaacttccatg

SEQ ID NO: 7 - DNA, 20 bp, synthetic construct
  source 1..20: mol_type = other DNA; note = Forward primer pBAD-F
cggcgtcacactttgctatg

SEQ ID NO: 8 - DNA, 21 bp, synthetic construct
  source 1..21: mol_type = other DNA; note = Reverse primer pBAD-R
cgtttcacttctgagttcggc

SEQ ID NO: 9 - DNA, 40 bp, synthetic construct
  source 1..40: mol_type = other DNA; note = Forward primer pTarget-fadDp-F
acgacgaacacgcattttaggttttagagctagaaatagc

SEQ ID NO: 10 - DNA, 40 bp, synthetic construct
  source 1..40: mol_type = other DNA; note = Reverse primer pTarget-fadDp-R
ctaaaatgcgtgttcgtcgtactagtattatacctaggac

SEQ ID NO: 11 - DNA, 40 bp, synthetic construct
  source 1..40: mol_type = other DNA; note = Forward primer pTarget-fadR-F
gctggctaccgctaatgaaggttttagagctagaaatagc

SEQ ID NO: 12 - DNA, 40 bp, synthetic construct
  source 1..40: mol_type = other DNA; note = Reverse primer pTarget-fadR-R
cttcattagcggtagccagcactagtattatacctaggac

SEQ ID NO: 13 - DNA, 20 bp, synthetic construct
  source 1..20: mol_type = other DNA; note = Forward primer fadD-up500-F
attaaaggcagcagtcccac

SEQ ID NO: 14 - DNA, 33 bp, synthetic construct
  source 1..33: mol_type = other DNA; note = Reverse primer fadD-up500-R
tataaggagggctgtttctttttctttaaaaac

SEQ ID NO: 15 - DNA, 33 bp, synthetic construct
  source 1..33: mol_type = other DNA; note = Forward primer CPA1- fadD-F
aagaaacagccctccttataacttcgtataatg

SEQ ID NO: 16 - DNA, 32 bp, synthetic construct
  source 1..32: mol_type = other DNA; note = Reverse primer CPA1- fadD-R
ccttcttcatgatatctccttcgtaaaagatc

SEQ ID NO: 17 - DNA, 31 bp, synthetic construct
  source 1..31: mol_type = other DNA; note = Forward primer fadD-down500-F
aggagatatcatgaagaaggtttggcttaac

SEQ ID NO: 18 - DNA, 20 bp, synthetic construct
  source 1..20: mol_type = other DNA; note = Reverse primer fadD-down500-R
tcggcaccaaacgcttgatg

SEQ ID NO: 19 - DNA, 20 bp, synthetic construct
  source 1..20: mol_type = other DNA; note = Forward primer fadR -up500-F
acttcaagatttgccgccac

SEQ ID NO: 20 - DNA, 32 bp, synthetic construct
  source 1..32: mol_type = other DNA; note = Reverse primer fadR -up500-R
gaatggctaacatagtgagatttccataacac

SEQ ID NO: 21 - DNA, 30 bp, synthetic construct
  source 1..30: mol_type = other DNA; note = Forward primer fadR -down500-F
tctcactatgttagccattcaggggcgata

SEQ ID NO: 22 - DNA, 20 bp, synthetic construct
  source 1..20: mol_type = other DNA; note = Reverse primer fadR -down500-R
gatatcgccggttccgactg

SEQ ID NO: 23 - DNA, 20 bp, synthetic construct
  source 1..20: mol_type = other DNA; note = Forward primer fadR-up700-F
tgtcttcggtacgggaagag

SEQ ID NO: 24 - DNA, 20 bp, synthetic construct
  source 1..20: mol_type = other DNA; note = Reverse primer fadR-down700-R
ggcactacaccatccttaac

SEQ ID NO: 25 - DNA, 20 bp, synthetic construct
  source 1..20: mol_type = other DNA; note = Forward primer fadD-up700-F
taaaacggtggcggtggaac

SEQ ID NO: 26 - DNA, 20 bp, synthetic construct
  source 1..20: mol_type = other DNA; note = Reverse primer fadD-down700-R
gtcgcgttaacctgttccag